(19) United States
(12) Patent Application Publication
(10) Pub. No.: US 2010/0130441 A1
(43) Pub. Date: May 27, 2010

(54) USE OF VITAMIN B12 FOR TREATMENT OF MUCOSAL LESIONS

(76) Inventors: Inna Rudoy, Beer-Sheva (IL); Ilia Volkov, Lehavim (IL); Yan Press, Beer-Sheva (IL)

Correspondence Address: BAKER BOTTS L.L.P., 30 ROCKEFELLER PLAZA, 44TH FLOOR, NEW YORK, NY, 10112-4498, US

(21) Appl. No.: 12/618,379

(22) Filed: Nov. 13, 2009

Related U.S. Application Data
(63) Continuation of application No. PCT/IB2008/003376, filed on May 14, 2008.

(30) Foreign Application Priority Data
May 14, 2007 (IL) 183171

(51) Int. Cl. A61K 31/714; A61P 15/02; A61P 11/02; A61P 1/04

(52) U.S. Cl. 514/52

(57) ABSTRACT

The present invention relates to the use of the nutrition supplement known as vitamin B12 (cobalamin) to treat or prevent mucosal lesions. It relates particularly to treatment of mucosal lesions and prevention of development of recurrent mucosal lesions by the repeated administration over a period of time of vitamin B12 where a subject has normal, or above normal, serum vitamin B12 levels. Compositions (medicaments), including friendly carriers (e.g. chewing gum), and kits, for vitamin B12 delivery to mucosal lesions or other injuries are also provided.

### CROSS-REFERENCE TO RELATED APPLICATIONS

This application is a continuation of International Application PCT/IB2008/003376, filed May 14, 2008, which claims priority under 35 U.S.C. §119 to Israel Application No. 183171 filed May 14, 2007, the contents of each of which are hereby incorporated herein by reference in their entirety.

### 1. INTRODUCTION

The present invention relates to the use of the nutrition supplement known as vitamin B12 (cobalamin) to treat or prevent mucosal lesions. It relates particularly to treatment of mucosal lesions and prevention of development of recurrent mucosal lesions by the repeated administration over a period of time of vitamin B12 where a subject has normal, or above normal, serum vitamin B12 levels. Compositions (medicaments), including friendly carriers (e.g. chewing gum), and kits, for vitamin B12 delivery to mucosal lesions or other injuries are also provided.

### 2. BACKGROUND OF THE INVENTION

Multifunctional systems have to maintain homeostasis. Man is an ideal example of a system that constantly aspires to attain optimal regulation, even under the stress of severe pathology. We assume that there are universal, interchangeable (as required) biologically active substances that regulate the system and try to keep it in balance. We have proposed that one of these substances is vitamin B12.

In a case report (Volkov et al., 2005 Jun. 10, “Case Report: Recurrent aphthous stomatitis responds to vitamin B12 treatment,” Canadian Family Physician 51(6), 844-845) the authors' key point was that when patients have recurrent aphthous ulcers, consider checking vitamin B12 levels and treating patients if levels are low. Such treatment could reduce some varieties of recurrent aphthous ulcers. All three patients from the case study above had low levels of serum vitamin B12.

Vitamin B12 administration led to rapid improvement and complete recovery within several weeks. Six months' follow up of all three patients revealed no recurrence of RAS. Thus, determining patients' vitamin B12 levels, as replacement therapy with this vitamin could be of considerable benefit to them.

The important role played by vitamin B12 in bodily processes is becoming increasingly clear as its involvement in a broad range of organs and systems is recognized and documented. It affects normal growth and development in children, the peripheral and central nervous systems, bone marrow, bones, skin, mucous membranes and vessels. Is there a “normal” serum level of vitamin B12? When have we attained the “correct” level? There are no generally accepted guidelines for the definition, diagnosis, treatment and follow-up of vitamin B12 (cobalamin) deficiency.

Total serum vitamin B12 may not reliably indicate vitamin B12 status. To get more specificity and sensitivity in diagnosing vitamin B12 deficiency, the concept of measuring homocystein (HCY), methylmalonic acid (MMA), holotranscobalamin II (holoTC, a sub-fraction of vitamin B12), has aroused great interest. HoloTC as a biologically-active vitamin B12 fraction promotes a specific uptake of its vitamin B12 by all cells (Volkov et al., 2005, “Successful Treatment of Chronic Erythema Nodosum with Vitamin B12,” The Journal of the American Board of Family Practice 18, 567-569). However, diagnostic algorithms using vitamin B12, MMA, and HCY measurements reflect studies in some academic centers, and their negative predictive values have not been established, therefore this problem remains controversial.

The measurements of vitamin B12 are quite accurate for serum vitamin B12 levels below 100 pg/ml, however, they discriminate poorly when vitamin B12 levels are between 100 and 400 pg/ml. The recommended cut-off for vitamin B12 deficiency is 250 pg/ml (normal range is >250 pg/ml or 185 μmol/L—Laboratory reference values (N Engl J Med 2004 Oct. 7; 351(15):1548-63).

The probability of “functional” vitamin B12 deficiency decreases with increasing level of vitamin B12. In order to explain the disappearance of generalized hyperpigmentation, erythema nodosum and recurrent canker sores as a result of vitamin B12 treatment, we suggest that vitamin B12 treatment effective because the B12 level is corrected or for other reasons.

It is conceivable that even if the serum level of cobalamin is within normal limits, treatment with vitamin B12 could correct defects caused by other biologically active substances. We have called this potential benefit the “Master Key” effect (Volkov et al., 2006 April, “Vitamin B12 could be a “Master Key” in the regulation of multiple pathological processes,” Journal of Nippon Medical School 73(2), 65-69). It is important to emphasize that vitamin B12 has no known significant toxic effects and has a low cost-effectiveness ratio.

There remains a need in the art for the treatment and prevention of mucosal lesions. The invention addresses this and other needs.

### 3. SUMMARY OF THE INVENTION

This invention provides for the use of vitamin B12 in the manufacture of a medicament for the treatment and/or prevention of mammalian mucosal lesions and/or injuries. Such lesions include recurrent aphthous stomatitis (RAS), also known as canker sores. Such use of B12 is without regard for the serum (plasma) level of vitamin B12 in the subject being treated. In particular, the level of B12 need not be below normal in the subject being treated, and can be within the normal range, or above the normal range. This use of B12 is also without regard for the specific cause of the mucosal lesion or injury, or any precipitating event or circumstance, such as stress or immunosuppression.

This invention provides user-friendly vitamin B12 carriers for the treatment or prevention of mucosal lesions. Such carriers include but are not limited to, chocolate bars, sweets, chewing gum, condoms, nasal sprays, toothpaste, mouthwash, douches, or suppositories. Such carriers are also referred to as “friendly carriers” herein and are intended to be included among the various compositions (medicaments) of the invention provided herein, in addition to conventional compositions (such as tablets or capsules), for delivery of vitamin B12 in the practice of the invention.

Such compositions, medicaments, and/or carriers contain an effective amount of vitamin B12 for the treatment and/or prevention of a mucosal lesion. They can be conveniently and repeatedly delivered to a mucosal membrane in need of treatment.

Virtually any mammalian mucosal membrane can be treated with B12 according to the invention. For example, the mammalian mucosal membranes that can receive this vitamin B12 therapy include but are not limited to, mucosae of the mouth, nose, vagina, anus, rectum, or other mucosal membrane of the gastrointestinal tract.

This invention provides for the repeated use, over a period of time, of the same user-friendly vitamin B12-containing composition, medicaments, and/or carriers, such as but not limited to, chocolate bar, sweets, chewing gum, condom, nasal spray, toothpaste, mouthwash, douche, suppository, tablet, or capsule, for preventing recurrence of mucosal lesions or injury.

In one embodiment, the invention provides for the use of an effective dosage of vitamin B12 for the treatment of buccal, nasal, gastrointestinal, anal, and/or vaginal mucosal lesions or injury of any origin, regardless of serum vitamin B12 level.

In another embodiment, the invention provides for the use of an effective dosage of vitamin B12 over a period of time to prevent recurring buccal, nasal, gastrointestinal, anal, and/or vaginal mucosal lesions or injury of any origin, regardless of serum vitamin B12 level.

The effective dosage of vitamin B12 is delivered to a human or other mammal in a convenient carrier such as a chocolate bar, sweets, biscuits, chewing gum, condom, nasal spray, toothpaste, mouthwash, douche, and/or suppository delivered to the mucosal membrane of the human or other mammal.

More specifically, this invention provides for the use of an effective dosage of vitamin B12 delivered to a mucosal membrane of a mammal for treatment or prevention of one or more of a buccal, nasal, gastrointestinal or vaginal mucosal lesion, or other mucosal injury of any origin, wherein the vitamin B12 level in the mammal before administration of the effective dosage is within a normal range or above a normal range.

In one embodiment, the mammal is a human and the effective dosage of vitamin B12 is delivered to vaginal mucosa by suppository, ointment, cream, or douche. In another embodiment, the effective dosage of vitamin B12 is delivered to the buccal mucosa of a mammal by dissolution in the mouth of any one or more of a chocolate bar, a biscuit, a tablet, a chewing gum, a sweet, a sublingual dot or drop, a lozenge, a toothpaste, a mouthwash, an ointment, or a wafer. In another embodiment, the effective dosage of vitamin B12 is delivered to the nasal mucosa of the mammal by any one or more of a nasal spray, a nasal aerosol, a nasal drop, or a salve. In another embodiment, the effective dosage of vitamin B12 is delivered to the anal mucosa of the mammal by any one or more of an anal suppository, an anal spray, an ointment, a drop, or a salve. In another embodiment, the mammal is a human and the buccal mucosal lesion or buccal mucosal injury is recurring aphthous stomatitis (RAS), also known as a recurring canker sore.

In another embodiment, an effective dosage of vitamin B12 is provided in a form suitable for treatment of a mammal having a mucosal lesion or mucosal injury, wherein the effective dosage is formulated in a carrier selected from the group consisting of a chocolate bar, a sweet, a biscuit, a chewing gum, a condom, a nasal spray, a toothpaste, a mouthwash, a douche, an ointment, a suppository, a tablet, and a capsule.

In another embodiment, an effective dosage of vitamin B12 is provided in a form suitable for delivery to a mammal for the prevention of a mucosal lesion or mucosal injury, wherein the effective dosage is formulated in a carrier selected from the group consisting of a chocolate bar, a sweet, a biscuit, a chewing gum, a condom, a nasal spray, a toothpaste, a mouthwash, a douche, an ointment, a suppository, a tablet, and a capsule.

This invention provides a kit comprising (i) a tablet or capsule comprising an effective amount of vitamin B12 suitable for the treatment or prevention of a mucosal lesion (e.g. a canker sore) or other mucosal injury, and (ii) instructions for use of the tablet or capsule in a subject having a plasma vitamin B12 level that is within or above a normal range. In some embodiments, the subject is a human and the normal range of plasma vitamin B12 is from 200 to 900 pg/mL.

This invention provides a kit comprising (i) a composition comprising an effective amount of vitamin B12 suitable for the treatment or prevention of a mucosal lesion (e.g. a canker sore) or other mucosal injury, and (ii) instructions for use of the composition in a subject having a plasma vitamin B12 level that is within or above a normal range. In some embodiments, the effective amount of vitamin B12 is formulated for intravenous or intramuscular administration. In some embodiments, the subject is a human and the normal range of plasma vitamin B12 is from 200 to 900 pg/mL.

### 4. BRIEF DESCRIPTION OF THE FIGURES

FIG. 1 shows the frequency of RAS episodes prior to and during vitamin B12 treatment (episodes per month).

### 5. DETAILED DESCRIPTION OF THE INVENTION

In various embodiments of the invention, the present invention provides a method for preventing or treating a mucosal lesion in a mammal in need of such prevention or treatment, comprising administering an effective amount of vitamin B12 to a mucosal membrane of the mammal, wherein the serum vitamin B12 level of the mammal is within or above the normal range.

A mucosal lesion or injury to be treated or prevented in accordance with the invention can occur on virtually any of the mucosal membranes of a mammal that is susceptible to such lesions or injury, including but not limited to a buccal mucosal membrane (e.g., inside the mouth or on or near the lips), a nasal mucosal membrane (e.g., inside the nose or at the edge of a nostril), a gastrointestinal mucosal membrane (e.g. gastric mucosa, duodenal mucosa, jejunal mucosa, ileal mucosa, cecal mucosa, colonic mucosa, rectal mucosa, or anal mucosa), a vaginal mucosal membrane, and a tracheal mucosal membrane. In some embodiments, a gastric ulcer is treated or prevented by the administration of an effective amount of vitamin B12 in accordance with the invention.

The vitamin B12 to be delivered to a mucosal membrane is typically formulated into daily-use or other frequent-use household items that are appropriate to and convenient for the particular mucosal membrane to be treated. For example, the vaginal mucosal membrane can be treated using a suppository, an ointment, a cream, or a douche containing B12. The buccal mucosal membrane can be treated, for example, by formulating B12 into a chocolate bar, a biscuit, a tablet, a chewing gum, a sweet, a sublingual drop, a lozenge, a toothpaste, a mouthwash, an ointment, a wafer, or any other like carrier.

For the nasal mucosa, vitamin B12 can be delivered using, for example, a nasal spray, a nasal aerosol, a nasal drop, or a salve. Vitamin B12 can be delivered to the anal mucosa, for example, using an anal suppository, an anal spray, an ointment, a drop, or a salve.

In a preferred embodiment of the invention, vitamin B12 is delivered in an effective amount to treat a recurrent canker sore, also known as a recurring aphthous stomatitis (RAS). In another preferred embodiment, vitamin B12 is delivered regardless of the serum level of B12 in the subject. For example, the subject can have a serum level of B12 that is within or above the normal range.

A human subject receiving a medicament containing vitamin B12 according to the invention will typically have a level of serum vitamin B12 within or above a normal range of, e.g., from 100 to 1800 pg/ml, from 200 to 900 pg/ml, from 250 to 500 pg/ml, or greater than 250 pg/ml.

### Effective Amount

An effective amount of vitamin B12 for use in accordance with the methods and compositions of the invention is provided. Generally, the effective amount can be higher than a B12 amount useful for nutritional supplementation or B12 replacement therapy, and, in accordance with the invention, is administered to subjects having a normal level, or an elevated level, of serum B12. As used herein, reference to a “serum” or “plasma” level of vitamin B12 is intended to be synonymous.

Accordingly, examples of effective amounts of vitamin B12 which can be used in accordance with the invention include but are not limited to the following: (i) intramuscular or intravenous injection of vitamin B12 at 100, 200, 400, 750, 1000, 2000, 3000, or 5000 mcg weekly for the first month, and then 100, 200, 400, 750 1000, 2000, 3000, or 5000 mcg monthly as maintenance therapy, as needed, for one or more months (e.g., for 2, 3, 4, 5, 6, 7, 8, 9, 10, 11, or 12 additional months); or (ii) one, two, or three vitamin B12 administrations per day of 50, 100, 200, 400, 750 1000, 2000, 3000, or 5000 mcg, as needed, until the mucosal lesion or injury is healed, or to prevent recurrence of the lesion, e.g. for 1, 2, 3, 4, 5, 6, 7, 8, 9, 10, 11, or 12 days, weeks, months, or even years. As noted below, there is no known toxic effect of prolonged B12 therapy, even at relatively high doses. B12 administration can be, for example, by injection, orally (e.g., by conventional tablet or capsule), or by other convenient form as described herein (e.g., chewing gum, sweet, toothpaste, mouthwash, etc.), or by convenient form directed to a particular mucosa (e.g., vaginal mucosa targeted by condom, douche, etc.).

The information below summarizes various studies on vitamin B12 therapy which have been performed for other indications and provides additional guidance on acceptable dosage, route, and administration of B12. The same or similar regimens are contemplated as providing effective amounts of vitamin B12 useful for the treatment and/or prevention of a mucosal lesion or injury in accordance with the invention. Moreover, for the avoidance of doubt, it is also contemplated that the same or similar dosage routes and timing of regimens can be used in the methods of the invention for the treatment or prevention of virtually any mucosal lesion or injury.

Thus, for example, without limitation, the Health and Consumer Protection Directorate-General of the European Commission (EC) has expressed the official opinion of the Scientific Committee on Food on the tolerable upper intake level of vitamin B12, and found no adverse effects associated with excess vitamin B12 intake from food or supplements in healthy individuals (Opinion expressed 19 Oct. 2000, published 28 Nov. 2000 by the EC, which is incorporated-by-reference herein in its entirety). In brief, due to the absence of clearly defined adverse effects produced by vitamin B12, no upper level has been derived to date. Indeed, vitamin B12 has a history of safe long-term use as a therapeutic agent given in high dosages per os (by mouth), or via intramuscular injections, for treatment of disorders associated with impaired vitamin B12 absorption, such as in gastrectomy and malabsorption.

As another example, in vitamin B12 replacement therapy, oral or intramuscular dosages between 1-5 mg vitamin B12 have been typically used, with no supportive evidence of adverse effects. Thus, the usual treatment in pernicious anemia patients is 1 mg (1000 mcg) administered intramuscularly once every 1 to 3 months, but oral dosages of 300-1000 mcg daily could also provide adequate treatment (Berlin et al., 1968, Oral treatment of pernicious anemia with high doses of vitamin B12 without intrinsic factor, Acta Med Scand 184, 247-258; Hathcock & Troendle, 1991, Oral cobalamin for treatment of pernicious anemia?, JAMA 265, 96-97).

As another example, Mangiarotti (Mangiarotti et al., 1986, Hypervitaminosis B12 in maintenance hemodialyse patients receiving massive supplementation of vitamin B12, Int J Artif Organs 9, 417-420) studied the effect of massive supplementation with vitamin B12 in a group of dialysis patients. One group of 106 patients received a multivitamin preparation containing 2.5 mg vitamin B12 plus 0.7 mg folic acid, 12 mg niacin and 150 mg vitamin C at the end of each dialysis period during 3 years. Serum vitamin B12 levels at the end of the treatment period were 4 times greater than normal, but no adverse effects were reported. As yet another example, high dosages have also been used in other experimental studies, mostly short term, such as for the treatment of sleep-waking rhythm disorders, in which study vitamin B12 (no form specified) was given in dosages between 1.5 and 3 mg/day for 8 weeks (n=13 cases), with no adverse effects recorded (Maeda et al., 1992, A multicenter study of the effects of vitamin B12 on sleep-waking rhythm disorders, In Shizuoka Prefecture, Japanese Journal of Psychiatry and Neurology 46, 229-231).

As yet another example, high dose vitamin B12 (1 mg cyanocobalamin intramuscular weekly for 1 months, followed by monthly injections for a minimum of 6 months), has also been used to improve cognitive functions in geriatric patients (Martin et al., 1992, Time dependency of cognitive recovery with cobalamin replacement: report of a pilot study, J Am Geriatr Soc 40, 168-172). Cobalamin therapy resulted in cognitive recovery in some patients, and no adverse effects were reported.

In some studies, intravenous (i.v.) dosing has been associated with dermal abnormalities, e.g. acne formation in some cases. Ten cases were reported by Puissant (Puissant et al., 1967, Bull Soc Fr Dermatol Syphiligr 74, 813-815) after series of up to 12 injections with 5 mg of hydroxocobalamin, but not with cyanocobalamin. These authors suggested that degradation products, formed from the less stable hydroxocobalamin, might have been responsible for the acne formation, rather than the intact compound.

One case of acneiform eruption, described as acne rosacea, was reported for a 53 year old women who used vitamin supplements containing 100 mg vitamin B6 and 100 mcg vitamin B12 and 10,000 IU vitamin A and an unknown amount of zinc.

Upon discontinuation of the supplement a ‘dramatic’ improvement was observed. The author ascribed the acne to the vitamins B6/B12 without further testing (Sheretz, 1991, Acneiform eruption due to megadose of vitamins B6 and B12, Cutis 48, 119-120).

Hydroxocobalamin is used as a cyanide antidote and also has a history of safe and effective use. For this purpose, intravenous dosages up to 5 g are given (Forsyth et al., 1993, Hydroxocobalamin as a cyanide antidote: safety, efficacy and pharmacokinetics in heavily smoking normal volunteers, J Toxicol Clin Toxicol 31, 277-294). Foulds (Foulds et al., 1969, The optic neuropathy of pernicious anemia, Arch Ophtal 82, 427-33) described four pernicious anemia patients presenting with tobacco amblyopia (optic neuropathy) who were treated parenterally with 0.25-1 mg cyanocobalamin per month. This treatment restored the haematological, but not the visual abnormalities. When the treatment was changed to hydroxocobalamin the visual impairment also improved. Cyanide, derived from tobacco smoke, has been implicated in the pathogenesis of tobacco amblyopia, and the positive effect of hydroxocobalamin is likely explained by cyanide detoxification.

No systematic toxicological studies have been reported for vitamin B12. There are no reports attributing carcinogenic or mutagenic or teratogenic potential to cyanocobalamin (Ellenbogen & Cooper, 1991, Vitamin B12, in Handbook of vitamins, 2nd Ed., Machlin L J, editor; Marcel Dekker Inc, pp 491-536). Oral and parenteral supplementation with dosages between 1-5 mg every fortnight or month have been given for long periods, up to at least 5 years, to patients with compromised vitamin B12 absorption, without any identified adverse effects.

Average intakes of vitamin B12 are about 2 to 6 mcg/day from food; intakes up to 32 mcg/day have been reported for the total intake (including supplements) in elderly Dutch subjects (van Asselt et al., 1998, Role of cobalamin intake and atrophic gastritis in mild cobalamin deficiency in older Dutch subjects, Am J Clin Nutr 68, 328-334). For the UK (HMSO, 1990, The Dietary and Nutritional survey of British Adults, ISBN 0 11 691300 2, HMSO Books, London) upper intake levels (97.5th percentile) from food sources only were reported to be 22.9 and 17.8 mcg/day, for males and females, respectively. Data from the United States show 95th percentile intakes from food and supplements of 83 mcg/day in elderly men, 106 mcg/day in elderly women, and 37 mcg/day in pregnant women.

Although it has not been possible to derive an upper limit of B12, there is no evidence that the current levels of intake from foods and supplements represent a health risk. In addition, adverse effects have not been reported in the treatment of patients with compromised B12 absorption who received dosages up to 1000 mcg/day orally for prolonged periods; however, there was no systematic assessment of adverse effects in these patients. Supplements available on the market usually contain dosages between 1-5 mcg, but higher dose supplements with 50 mcg or more are also available. Such supplements can be used in accordance with the invention.

The foregoing studies on vitamin B12 replacement therapy are for indications other than mucosal lesion or injury. Nevertheless, these indications serve as appropriate examples of additional guidance to the skilled artisan for providing a levels of B12 effective for the treatment or prevention of a mucosal lesion or injury in accordance with the invention.

Additional information is provided in the following references: Adams et al., 1972, Interrelation of serum vitamin B12, total body vitamin B12, peripheral blood morphology and the nature of erythropoiesis, Br J Haematol 23, 297-305; Blokdijk et al., 2000, Voeding Nu 2, 13-15; Bower et al., 1995, Review: vitamin B12 deficiency and the fortification of food with folic acid, E J Clin Nutr 49, 787-793; Grasbeck et al, 1958, Biliary and fecal vitamin B12 excretion in man, An isotope study, Proc Soc Exp Biol Med 97, 780-784; Herbert, 1984, Vitamin B-12, In, Nutrition reviews, Present Knowledge in Nutrition, The Nutrition Foundation Inc, Washington D.C., pp 347-64; Herbert, 1987, The 1986 Herman Award lecture, Nutrition science as a continually unfolding story: the folate and vitamin B-12 paradigma, Am J Clin Nutr 46, 387-402; IUNA, 2000, Irish Universities Nutrition Alliance, The North/South Ireland Food Consumption Survey, Food Safety Promotion Board, Dublin; Lindenbaum et al., 1988, Neuropsychiatric disorders caused by cobalamin deficiency in the absence of anemia or macrocytosis, N Engl J Med 318, 1720-1728; Loew, 1988, Pharmacokinetics of hydroxocobalamins and folic acid, Vitaminspur 3, 168-172; Raccuguglia et al., 1969, Absorption and excretion of cyanocobalamin after oral administration of a large dose in various conditions, Acta Haemat 42, 1-7; Report of the Standing Committee on the scientific evaluation of dietary reference intakes and its panel on folate and other B-vitamins and choline, Food and Nutrition Board, Institute of Medicine, National Academy Press, Washington, D.C., 1998; and Scott, 1997, Bioavailability of vitamin B12, Eur J Clin Nutr 51, S49-S53.

### Formulation and Administration

In addition to a “friendly carrier” as already described herein (e.g., a chewing gum, a sweet, a toothpaste, a mouthwash, a condom, etc.), the vitamin B12 can also be formulated into a conventional pharmaceutical composition or medicament together with any number of other pharmaceutically acceptable carriers. Such a pharmaceutical composition (which is synonymous with a medicament herein) is also simply referred to as a composition herein. Further, such compositions of the invention are intended to include the friendly carriers already described.

The concentration or amount of the active ingredient (vitamin B12) is discussed above and depends on the desired dosage regimen and administration regimen. Without limitation, further exemplary dose ranges of vitamin B12 are from about 50 μg to about 5000 μg per day; from about 100 μg to about 2500 μg per day; from about 200 μg to about 1500 μg per day; or from about 250 μg to about 1000 μg per day. Alternatively, dose ranges of the active ingredient are from about 50 μg to about 2500 μg weekly; from about 100 μg to about 2000 μg weekly; from about 300 μg to about 1500 μg weekly; or from about 500 μg to about 1000 μg weekly for a first month, and then a similar amount (e.g., about 1000 μg) monthly as maintenance therapy. The concentration or effective amount of the active ingredient can be modified so as to produce a desired therapeutic response, for example, any response that a user (e.g., a clinician) will recognize as an effective response to the therapy. A therapeutic response will generally be an amelioration of one or more symptoms of a disease or disorder, specifically, clinical judgment or evidence of healing activity in a mucosal lesion or injury. As one example, the disappearance of a canker sore can indicate the effectiveness of the therapeutic regimen.

Therapeutically effective amounts of B12 for the treatment or prevention of mucosal lesion or injury can be provided to a subject in virtually any formulation. Standard formulations are well known in the art. See e.g., Remington: The Science and Practice of Pharmacy, Lippincott Williams & Wilkins; 21 edition (May 1, 2005). Vitamin B12 can be administered in a form suitable for any route of administration, including, e.g., orally in the form tablets, capsules, or liquid (e.g., lingual drops or gastric coating formulations), or in sterile aqueous solution for injection. For example, vitamin B12 can be administered orally in the form of tablets, capsules, ovules, elixirs, solutions or suspensions, gels, syrups, mouth washes, or a dry powder for constitution with water or other suitable vehicle before use, optionally with flavoring and coloring agents, formulated for immediate-, delayed-, modified-, sustained-, pulsed-, or controlled-release applications.

Solid compositions such as tablets or capsules (including e.g. slow-release forms), lozenges, pastilles, pills, boluses, powders, pastes, granules, bullets, or premix preparations may also be used. Solid and liquid compositions for oral use may be prepared according to methods well known in the art. Such compositions may also contain one or more pharmaceutically acceptable carriers and excipients which may be in solid or liquid form. When the compound is formulated for oral administration, the tablets or capsules can be prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art.

Pharmaceutically acceptable excipients also include microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrolidone, hydroxypropyl ethylcellulose (HPMC), hydroxypropyl cellulose (HPC), sucrose, gelatin, and acacia. Additionally, lubricating agents such as magnesium stearate, stearic acid, glyceryl behenate and talc may be included.

Solid compositions may also be employed as fillers in gelatin capsules. Preferred excipients in this regard include lactose, starch, a cellulose, milk sugar, or high molecular weight polyethylene glycols. For aqueous suspensions and/or elixirs, the agent may be combined with various emulsifying and/or suspending agents and with diluents such as water, ethanol, propylene glycol and glycerin, and combinations thereof.

Liquid preparations for oral administration may take the form of, for example, solutions, syrups or suspensions, or they may be presented as a dry product for constitution with water or another suitable vehicle (for example, ethanol or a polyol such as glycerol, propylene glycol, and polyethylene glycol, and the like, suitable mixtures thereof, and vegetable oils) before use. Such liquid preparations may be prepared by conventional means with pharmaceutically acceptable additives such as suspending agents (e.g., water, sorbitol syrup, cellulose derivatives or hydrogenated edible fats); emulsifying agents (e.g., lecithin or acacia); non aqueous vehicles (e.g., almond oil, oily esters, ethyl alcohol or fractionated vegetable oils); and preservatives (e.g., methyl or propyl-p-hydroxybenzoates or sorbic acid). Preparations for oral administration may be suitably formulated to give a controlled release or sustained release of vitamin B12.

The proper fluidity can be maintained, for example, by the use of a coating such as lecithin, by the maintenance of the required particle size in the case of dispersion and by the use of surfactants. Prevention of the action of microorganisms can be brought about by various antibacterial and antifungal agents, for example, parabens, chlorobutanol, phenol, benzyl alcohol, sorbic acid, and the like. Prolonged absorption of a injectable composition can be brought about by the use in the compositions of agents delaying absorption, for example, aluminum monosterate, and gelatin.

The pharmaceutical formulations of vitamin B12 suitable for parenteral/injectable (for example, by intravenous bolus injection or infusion or via intramuscular, subcutaneous or intrathecal routes) use generally include sterile aqueous solutions, or dispersions and sterile powders for the extemporaneous preparation of sterile injectable solutions or dispersion. The vitamin B12 may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing, and/or dispersing agents. Alternatively, the active ingredient may be in sterile powder form for reconstitution with a suitable vehicle, e.g., sterile, pyrogen-free water, before use. The injectable form is sterile and fluid to the extent that easy syringability exists. It is stable under the conditions of manufacture and storage and can be preserved against the contaminating action of microorganisms such as bacteria and fungi. The preparation of suitable parenteral formulations under sterile conditions is readily accomplished by standard pharmaceutical techniques well known to those skilled in the art.

Injectable solutions are prepared by incorporating vitamin B12 in the required amount in the appropriate solvent with various of the other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

Preservatives, stabilizers, dyes, and even flavoring agents may be provided in the pharmaceutical composition. Examples of preservatives include sodium benzoate, ascorbic acid, and esters of p-hydroxybenzoic acid. Antioxidants and suspending agents may be also used.

Additional pharmaceutically acceptable carriers which may be included in a formulation are buffers such as citrate buffer, phosphate buffer, acetate buffer, and bicarbonate buffer, amino acids, urea, alcohols, ascorbic acid, phospholipids, proteins, such as serum albumin, collagen, and gelatin; salts such as EDTA or EGTA, and sodium chloride; liposomes; polyvinylpyrolidone; sugars such as dextran, mannitol, sorbitol, and glycerol; propylene glycol and polyethylene glycol (e.g., PEG-4000, PEG-6000); glycerol, glycine or other amino acids and lipids. Buffer systems for use with the formulations include citrate, acetate, bicarbonate, and phosphate buffers.

The formulations can also contain a non-ionic detergent. Preferred non-ionic detergents include Polysorbate 20, Polysorbate 80, Triton X-100, Triton X-114, Nonidet P-40, Octyl α-glucoside, Octyl β-glucoside, Brij 35, Pluronic, and Tween 20.

The routes for administration (delivery) include, but are not limited to, one or more of: oral (e.g., as a tablet, capsule, or as an ingestible solution), topical, mucosal (e.g., as a nasal spray or aerosol for inhalation), nasal, parenteral (e.g., by an injectable form), gastrointestinal, intraperitoneal, intramuscular, intravenous, intrauterine, intradermal, intravaginal, subcutaneous, transdermal, rectal, buccal, and sublingual.

Administration of the above-described parenteral formulations of vitamin B12 may be by periodic injections of a bolus of the preparation, or may be administered by intravenous or intraperitoneal administration from a reservoir which is external (e.g., an i.v. bag) or internal (e.g., a bioerodable implant). See, e.g., U.S. Pat. Nos. 4,407,957 and 5,798,113, each incorporated herein by reference. Other useful parenteral delivery systems include ethylene-vinyl acetate copolymer particles, osmotic pumps, implantable infusion systems, pump delivery, encapsulated cell delivery, liposomal delivery, needle-delivered injection, nebulizer, aeorosolizer, electroporation, and transdermal patch. Furthermore, a variety of devices designed for patient convenience, such as refillable injection pens and needle-less injection devices, can be used with formulations of the present invention.

### Kits

The invention further provides kits containing the various vitamin B12 compositions of the invention. Such kits can also be used to facilitate the convenient practice of the methods of the invention.

For example, without limitation, the invention provides a kit comprising (i) a tablet or capsule comprising an effective amount of vitamin B12 suitable for the treatment or prevention of a mucosal lesion (e.g. a canker sore) or any other mucosal injury, and (ii) instructions for use of the tablet or capsule in a subject having a plasma vitamin B12 level that is within or above a normal range. The subject can be any mammal. In some embodiments, the subject is a human and the normal range of plasma vitamin B12 is from 200 to 900 pg/mL. Thus, a kit can be a blister pak of tablets or capsules accompanied by instructions in accordance with the invention.

As another example, without limitation, this invention provides a kit comprising (i) a composition comprising an effective amount of vitamin B12 suitable for the treatment or prevention of a mucosal lesion (e.g. a canker sore) or any other mucosal injury, and (ii) instructions for use of the composition in a subject having a plasma vitamin B12 level that is within or above a normal range. The subject can be any mammal. In some embodiments, the subject is a human and the normal range of plasma vitamin B12 is from 200 to 900 pg/mL. In other embodiments, the effective amount of vitamin B12 is formulated for intravenous or intramuscular administration.

Various kits of the invention can be assembled to provide convenient, ready access for a subject in need of vitamin B12 treatment in accordance with the invention, or for a care provider (e.g. a clinician), for virtually any of the methods and compositions described herein. For example, a chewing gum, a sweet, a mouthwash, or a toothpaste can be provided in a kit together with instructions for use in the treatment or prevention of a mucosal lesion (e.g. a canker sore) or other mucosal injury (e.g. a scrape or burn). As another example, a kit can be a pack of chewing gum with instructions for periodic use (e.g. daily, weekly, as needed) written on the pack or placed within the pack for the treatment or prevention of a canker sore, or a stomach ulcer, where the chewing gum contains an effective amount of vitamin B12. The need for treatment can be indicated, for example, by the presence of pain from the canker sore, or the stomach ulcer, or virtually any other clinical sign or symptom associated with the mucosal lesion to be treated.

### 6. EXAMPLE

### Successful Treatment of Recurrent Aphthous Stomatitis (RAS) of any Origin with Vitamin B12 (Irrespective of B12 Blood Level)

Recurrent aphthous stomatitis (RAS) is one of the most common oral mucosal lesions seen in primary care. This example describes our clinical experience over a four year period in treating 15 RAS patients with vitamin B12. Of these, 11 patients reported a rapid and complete recovery from RAS during treatment and the other four reported pronounced reduction in the frequency and severity of RAS episodes. We hypothesized that a treatment with vitamin B12 can be effective for patients suffering from RAS any origin, regardless of their serum vitamin B12 level, and performed a double-blind, placebo-controlled, randomized clinical trial to address the issue. The clinical trial has established the safety and effectiveness of vitamin B12 therapy for mucosal lesions such as RAS.

### Introduction

Recurrent aphthous stomatitis (RAS) is one of the most common oral mucosa lesions seen in primary care (1, 2). The Greek team “aphthai” was initially used in relation to disorders of the mouth and is credited to Hippocrates (1). RAS is a pathologic condition characterized by recurring, painful, small, oral mucosal ulcers with a round or oval aspect, clean borders, a peripheral erythematous halo, and a yellow or grayish base (1, 3).

The frequency of aphthous ulcers is up to 25% in the general population, and three-month recurrence rates are as high as 50% (2). RAS is an idiopathic condition in most patients. The most likely precipitating factors are local trauma and stress. Other associated factors include systemic diseases, nutritional deficiencies, food allergies, genetic predisposition, immune disorders, medications, and HIV infection.

Although RAS may be a marker of an underlying systemic illness such as celiac disease, or may present as one of the features of Behcet's disease, in most cases no other body systems are affected, and patients remain otherwise fit and well. Since the etiology of RAS is unknown, diagnosis is entirely based on history and clinical criteria and no laboratory procedures exist to confirm the diagnosis (4, 5, 6, 7).

Herbal multivitamins (8), adhesive pastes (9), local antiseptics (10), local antibiotics (11), topical non-steroidal anti-inflammatory drugs (12), topical corticosteroids (13), and even topical and systemic immunomodulators, immunosuppressants, and corticosteroids (14, 15, 16) are among the treatments given to RAS patients. Most of these treatments achieve “short term” therapeutic goals such as alleviation of pain, reduction of ulcer duration, and recovery of normal oral function (9, 10, 11, 12, 13, 16). Very few treatments have been reported to achieve “long term” therapeutic goals such as reduction of the frequency and severity of RAS and maintenance of remission (8, 13, 14, 15, 16).

We previously reported the successful treatment of three RAS patients having low levels of serum vitamin B12 with intramuscular (IM) vitamin B12 injections (17). We have now treated 15 RAS patients with vitamin B12. In this example, we review our clinical experience with 15 RAS patients, and report the strongly positive outcome of a subsequent double-blind, placebo-controlled, randomized clinical trial. Results of this double blind, placebo control study, conducted in the primary care setting, indicate that vitamin B12 treatment can achieve the “long term” therapeutic goal of management of RAS in terms of various disease parameters including: pain, number of ulcers, and duration of outbreak. Patient outcome did not depend on initial level of vitamin B12. This treatment is simple and inexpensive and has no known significant toxic effects.

### Methods

Fifteen patients suffering from RAS were treated with vitamin B12 in our clinics over a period of four years. In most cases they have presented to the clinic with unrelated symptoms, and the oral ulcers were incidental findings on physical examination. Patients were asked whether oral ulcers were a recurring problem. Before initiating vitamin B12 therapy, a complete blood count was done and plasma vitamin B12 and folic acid levels were assessed by routine procedures.

We used one of two therapeutic regimens:
IM injections of vitamin B12 (1000 mcg weekly for the first month, and then 1000 mcg monthly as maintenance therapy) for patients with serum vitamin B12 level below 100 pg/ml, for patients with peripheral neuropathy or macrocytic anemia, and for patients coming from a low socioeconomic level (in Israel, IM vitamin B12 treatment is cheaper than oral vitamin B12).
2. One sublingual vitamin B12 tablet (1000 mcg) per day.
No other treatment was given for RAS throughout the treatment and follow-up periods. The follow-up period ranged from 3 months to 4 years.

### Results

This report presents the results of treatment for 15 patients from two primary care practices. Nine patients (60%) were males. The mean age was 38.7±18.8 years (range 15-86 years). The patient population was ethnically heterogenic with 8 Jewish and 7 Bedouin patients. The mean duration of RAS prior to vitamin B12 therapy was 11.2±10.7 years (range 1-38 years). The results of blood tests prior to therapy are shown in Table 1.

TABLE 1
Plasma hemoglobin, mean corpuscular volume (MCV), vitamin
B12 and folic acid levels in the study population

| | Mean ± SD | Range | Normal range* |
|---|---|---|---|
| B12 before treatment | 303.6 ± 169.5 | 88-816 | >250 |
| (pg/ml) | | | |
| Hemoglobin before | 13.6 ± 2.0 | 10.0-16.8 | Males: 13.5-17.5 |
| treatment (g/dl) | | | Females: 12-16 |
| MCV before treatment | 85.3 ± 6.9 | 75-104 | 80-100 |
| (fl) | | | |
| Folic acid before | 6.9 ± 3.5 | 1.5-12.3 | 3.1-17.5 |
| treatment (ng/ml) | | | |

*Ref 18

TABLE 2
RAS disease characteristics before and during
treatment, and length of treatment period

| | Mean | Range |
|---|---|---|
| Mean duration of RAS (years) | 11.2 ± 10.7 | 1-38 |
| Frequency of RAS before treatment | 1.5 ± 1.2 | 0.3-4.0 |
| (episodes/month) | | |
| Mean duration of treatment (months) | 10.8 ± 9.7 | 3-42 |
| Frequency of RAS during treatment | 0.1 ± 0.3 | 0-1.0 |
| (episodes/month) | | |

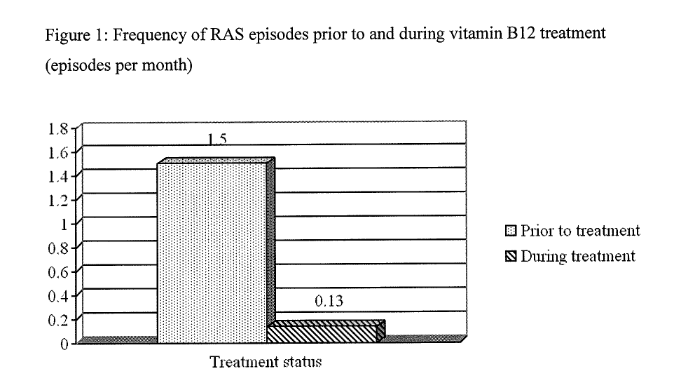

Eleven of the 15 patients (73%) were treated by IM injection, in most cases due to socioeconomic considerations. The main results of treatment are presented in Table 2 and FIG. 1. Eleven patients reported a rapid and complete recovery from RAS during treatment and the other four reported pronounced reduction in the frequency and severity of RAS episodes. Two of the four patients who did not report complete recovery were treated with sublingual vitamin B12. The other two patients, who were treated with IM vitamin B12, had long periods of non-adherence (over 2 months). When these two non-adherent patients received regular IM injections their aphthous ulcers disappeared completely.

### Discussion

In this example we review our clinic experience with 15 patients treated with vitamin B12 for RAS. Interestingly, none of the patients complained of oral ulcers; all cases were discovered only on physical examination. Reviewing the literature, we were amazed by statistics that 10 to 50% of the general population suffers from RAS, and up to 60% of the medical staff! Why were we surprised? RAS is not considered a reason to pay a visit to the primary physician. Patients rarely complain of RAS, except for how it influences their daily lives. When we started to elucidate the phenomenon, we understood that at some moment aphthae in one's mouth was accepted as “a part of life.” Many affected patients may not seek medical help because they don't believe that there is a definitive solution to their problem. Therefore, primary care physicians should actively inquire about this problem.

Although the precise role of vitamin B12 deficiency in the pathogenesis of RAS is unclear, suppression of cell-mediated immunity and changes in the cells of the tongue and buccal mucosa have been reported (18, 19). In terms of the normal range of vitamin B12 levels (20), only two patients in our patient population had vitamin B12 deficiency (below 125 pg/ml), three others had marginal levels (from 125 to 250 pg/ml) and the other 10 had normal levels (above 250 pg/ml). There was no difference in the outcome of treatment between patients with deficient or borderline levels vitamin B12 levels, and those with a normal level.

How can this phenomenon be explained? In most likelihood, a serum vitamin B12 level may not reliably indicate “functional” vitamin B12 status. The test for B12 has several pitfalls (21). Most laboratories set normal limits at 200 to 900 pg/mL, but sensitivity and specificity vary greatly, depending on the method used. False negatives (i.e., elevated levels in the presence of deficiency) can occur in true deficiency, active liver disease, lymphoma, autoimmune disease, and myeloproliferative disorders. False positives (i.e., low levels in the absence of deficiency) can occur in folate deficiency, pregnancy, multiple myeloma, and excessive vitamin C intake. The measurements are quite accurate for serum vitamin B12 levels below 100 pg/mL Some authors have suggested that vitamin B12 treatment should be offered to all patients with clinical appearances of vitamin B12 deficiency, even if their serum vitamin B12 level is normal (22).

Another explanation for this phenomenon (our “working hypothesis”) is that vitamin B12 has some unique, but still unrecognized functions. Multifunctional systems have to maintain homeostasis. Man is an ideal example of a system that constantly aspires to attain optimal regulation, even under the stress of severe pathology. We assume that there are universal, interchangeable (as required) biologically active substances that regulate the system and try to keep it in balance. We propose that one of these substances is vitamin B12.

Why vitamin B12? The list of organs and body systems in which vitamin B12 plays a functional role is constantly being added to. Vitamin B12 affects the normal growth of children, the peripheral and central nervous systems, bone marrow, skin and mucous membranes, bones, and vessels. Vitamin B12 (cobalamin) is unique among all the vitamins in that it contains not only a complex organic molecule but also an essential trace element, cobalt. Vitamin B12 plays an important role in DNA synthesis and has important immunomodulatory and neurotrophic effects. Deficiency of vitamin B12 can lead to a wide spectrum of disorders that can often be reversed by early diagnosis and prompt treatment.

It is possible that even when the serum cobalamin level is high, treatment with vitamin B12 can correct defects caused by other biologically active substances. We suppose this has been proved successful in the treatment of recurrent aphthous stomatitis with vitamin B12 (irrespective of its blood level !). We call this phenomenon the “Master Key” effect (23).

### CONCLUSION

Treatment with vitamin B12 can be effective for patients suffering from RAS of any origin, regardless of their serum vitamin B12 level. We have completed a double-blind, placebo-controlled, randomized clinical trial addressing the issue. This trial has confirmed the safety and effectiveness of vitamin B12 therapy for RAS.

### REFERENCES

1. Ship J A, Chavez E M, Doerr P A, Henson B S, Sarmadi M. Recurrent aphthous stomatitis. Quintessence Int 2000; 31:95-112.
2. Barrons R W. Treatment strategies for recurrent oral aphthous ulcers. Am J Health Syst Pharm 2001; 58:41-50.
3. Porter S R, Hegarty A, Kaliakatsou F, Hodgson T A, Scully C. Recurrent aphthous stomatitis. Clin Dermatol 2000; 18:569-78.
4. Piskin S, Sayan C, Durukan N, Senol M. Serum iron, ferritin, folic acid, and vitamin B12 levels in recurrent aphthous stomatitis. J Eur Acad Dermatol Venereol 2002; 16:66-7.
5. Wray D, Ferguson M M, Mason D K, Hutcheon A W, Dagg J H. Recurrent aphthae: treatment with vitamin B12, folic acid, and iron. BMJ 1975; 2:490-3.
6. Barnadas M A, Remacha A, Condomines J, de Moragas J M. [Hematologic deficiencies in patients with recurrent oral aphthae]. Med Clin (Barc) 1997; 109:85-7.
7. Natah S S, Konttinen Y T, Enattah N S, Ashammakhi N, Sharkey K A, Hayrinen-Immonen R. Recurrent aphthous ulcers today: a review of the growing knowledge. Int J Oral Maxillofac Implants 2004; 33:221-34.
8. Pedersen A, Hougen H P, Klausen B, Winther K. LongoVital in the prevention of recurrent aphthous ulceration. J Oral Pathol Med 1990; 19:371-5.
9. Reznik D, O'Daniels C M. Clinical treatment evaluations of a new topical oral medication. Compend Contin Educ Dent Suppl 2001; 32:17-21.
10. Meiller T F, Kutcher M J, Overholser C D, Niehaus C, DePaola L G, Siegel M A. Effect of an antimicrobial mouthrinse on recurrent aphthous ulcerations. Oral Surg Oral Med Oral Pathol Oral Radiol Endod 1991; 72:425-9.
11. Kerr A R, Drexel C A, Spielman A I. The efficacy and safety of 50 mg penicillin G potassium troches for recurrent aphthous ulcers. Oral Surg Oral Med Oral Pathol Oral Radiol Endod 2003; 96:685-94.
12. Murray B, McGuinness N, Biagioni P, Hyland P, Lamey P J. A comparative study of the efficacy of Aphtheal in the management of recurrent minor aphthous ulceration. J Oral Pathol Med 2005; 34:413-9.
13. Gonzalez-Moles M A, Morales P, Rodriguez-Archilla A, Isabel I R, Gonzalez-Moles S. Treatment of severe chronic oral erosive lesions with clobetasol propionate in aqueous solution. Oral Surg Oral Med Oral Pathol Oral Radiol Endod 2002; 93:264-70.
14. Hutchinson V A, Angenend J L, Mok W L, Cummins J M, Richards A B. Chronic recurrent aphthous stomatitis: oral treatment with low-dose interferon alpha. Mol Biother 1990; 2:160-4.
15. Katz J, Langevitz P, Shemer J, Barak S, Livneh A. Prevention of recurrent aphthous stomatitis with colchicine: an open trial. J Am Acad Dermatol 1994; 31(3 Pt 1):459-61.
16. Femiano F, Gombos F, Scully C. Recurrent aphthous stomatitis unresponsive to topical corticosteroids: a study of the comparative therapeutic effects of systemic prednisone and systemic sulodexide. Int J Dermatol 2003; 42:394-7.
17. Volkov I, Rudoy I, Abu-Rabia U, Masalha T, Masalha R. Recurrent apthous stomatitis responsive to vitamin B12 treatment. Can Fam Phys 2005; 51:844-5.
18. Field E A, Speechley J A, Rugman F R, Varga E, Tyldesley W R. Oral signs and symptoms in patients with undiagnosed vitamin B12 deficiency. J Oral Pathol Med 1995; 24:468-70.
19. Weusten B L, van de Wiel A. Aphthous ulcers and vitamin B12 deficiency. Neth J Med 1998; 53:172-5.

20. Kratz A, Ferraro M, Sluss P M, Lewandrowski K B. Case records of the Massachusetts General Hospital. Weekly clinicopathological exercises. Laboratory reference values. N Engl J Med 2004; 351:1548-63.
21. Dharmarajan T S, Norkus E P. Approaches to vitamin B12 deficiency. Early treatment may prevent devastating complications. Postgrad Med 2001 July; 110(1):99-105.
22. Solomon L R. Cobalamin-responsive disorders in the ambulatory care setting: unreliability of cobalamin, methylmalonic acid, and homocysteine testing. Blood 2005 Feb. 1; 105(3):978-85.
23. Ilia Volkov MD, Yan Press MD, Irma Rudoy MD. Vitamin B12 could be a “Master Key” in the regulation of multiple pathological processes. Journal of Nippon Medical School. 2006; 73(2): 65-69.
24. Ilia Volkov, Irma Rudoy, Roni Peleg, Yan Press. Successful treatment of recurrent aphthous stomatitis of any origin with vitamin B12 (irrespective of its blood level). The Internet Journal of Family Practice (ISSN: 1528-8358). 2007 (August). Volume 5, Number 1.

Various publications are cited herein, the contents of which are hereby incorporated by reference in their entireties for all purposes.

What is claimed is:

1. A method of preventing or treating a mucosal lesion in a mammal in need of such treatment, comprising administering an effective amount of vitamin B12 to a mucosal membrane of the mammal, wherein the plasma vitamin B12 level of the mammal is within or above a normal range.

2. The method of claim 1, wherein the mucosal membrane with the mucosal lesion or injury being prevented or treated is selected from the group consisting of a buccal mucosal membrane, a nasal mucosal membrane, a gastrointestinal mucosal membrane and a vaginal mucosal membrane.

3. The method of claim 2, wherein the mammal is a human and the effective amount of vitamin B12 is delivered to the vaginal mucosal membrane using a suppository, an ointment, a cream, a condom, or a douche.

4. The method of claim 2, wherein the effective amount of vitamin B12 is delivered to the buccal mucosal membrane using a chocolate bar, a biscuit, a tablet, a chewing gum, a sweet, a sublingual drop, a lozenge, a toothpaste, a mouthwash, an ointment, or a wafer.

5. The method of claim 2, wherein the effective amount of vitamin B12 is delivered to the nasal mucosal membrane using a nasal spray, a nasal aerosol, a nasal drop, or a salve.

6. The method of claim 2, wherein the gastrointestinal mucosal membrane is gastric mucosa, duodenal mucosa, jejunal mucosa, ileal mucosa, cecal mucosa, colonic mucosa, rectal mucosa, or anal mucosa.

7. The method of claim 1, wherein the mammal is a human and the mucosal lesion is a buccal mucosal lesion known as recurring aphthous stomatitis (RAS).

8. The method of claim 1, wherein the mammal is a human and the normal range of plasma vitamin B12 is from 200 to 900 pg/mL.

9. The method of claim 1 comprising periodic use of a composition comprising an amount of vitamin B12 effective for the treatment or prevention of the mucosal lesion or injury, wherein the plasma vitamin B12 level in the mammal is within or above a normal range.

10. The method of claim 1, wherein the cause of the lesion is selected from the group consisting of: stress, viral disease, and plasma iron levels below the normal range.

11. The method of claim 1, wherein the mammal is a human and the effective amount of vitamin B12 is provided to the human in a friendly carrier.

12. The method of claim 1, wherein the friendly carrier is a chocolate bar, a sweet, a chewing gum, a toothpaste, a mouthwash, a condom, or a douche.

13. The method of claim 1, wherein the mucosal lesion or injury is a canker sore, comprising administering an effective amount of vitamin B12 to a buccal mucosal membrane of the human, wherein the plasma vitamin B12 level of the human is within or above a normal range.

14. A composition comprising an amount of vitamin B12 effective for the prevention or treatment of a mucosal lesion in a mammal, wherein the effective amount of vitamin B12 is contained in a chocolate bar, a sweet, a biscuit, a chewing gum, a condom, a nasal spray, a toothpaste, a mouthwash, a douche, an ointment, or a suppository.

15. The composition of claim 14 wherein the mammal is a human.

16. A kit comprising (i) a composition comprising an effective amount of vitamin B12 suitable for the treatment or prevention of a canker sore, and (ii) instructions for use of the tablet or capsule in a subject having a plasma vitamin B12 level that is within or above a normal range.

17. The kit of claim 16, wherein the subject is a human and the normal range of plasma vitamin B12 is from 200 to 900 pg/mL.

18. The kit of claim 16 wherein the composition comprising an effective amount of vitamin B12 suitable for the treatment or prevention of a canker sore is a tablet or capsule.

19. The kit of claim 16, wherein the composition comprising an effective amount of vitamin B12 suitable for the treatment or prevention of a canker sore is formulated for intravenous administration.

20. The kit of claim 19, wherein the effective amount of vitamin B12 is formulated for intramuscular administration.
